(19) United States
(12) Patent Application Publication
(10) Pub. No.: US 2010/0304502 A1
(43) Pub. Date: Dec. 2, 2010

(54) DETECTION METHOD AND DETECTION KIT

(75) Inventors: Natsuki Ito, Tokyo (JP); Kazuya Tsukada, Kanagawa (JP)

Correspondence Address: LUCAS & MERCANTI, LLP, 475 PARK AVENUE SOUTH, 15TH FLOOR, NEW YORK, NY, 10016, US

(73) Assignee: KONICA MINOLTA MEDICAL & GRAPHIC, INC., Tokyo (JP)

(21) Appl. No.: 12/745,563

(22) PCT Filed: Nov. 28, 2008
(86) PCT No.: PCT/JP2008/071664

§ 371 Date: Jun. 1, 2010

(30) Foreign Application Priority Data
Dec. 5, 2007 (JP) 2007-315128

(51) Int. Cl. G01N 33/553

(52) U.S. Cl. 436/525

(57) ABSTRACT

A detection method for detecting a substance such as a virus which achieves enhanced detection sensitivity even in trace amounts of a substance to be detected and is also simple and superior in visual determinability is disclosed, comprising contacting a complex of a substance to be detected and a semiconductor nanoparticle-labeled probe capable of bonding the substance with an immobilized capture reagent capable of bonding the substance to detect the substance. A detection kit is also disclosed.

### TECHNICAL FIELD

The present invention relates to a detection method of a substance to be detected by use of semiconductor nanoparticles and in particular to a detection method by employment of an immunochromatography method, and a detection kit used therein. Specifically, the present invention relates to a method of detecting a virus such as an influenza virus by using a semiconductor nanoparticle-labeled probe and a detection kit thereof.

### TECHNICAL BACKGROUND

In practice, there has been realized a methodology such as an immuno-diffusion method, an enzyme measurement method, a coagulation method or the like, as a method for detecting or quantitative-determining a substance in a specimen by employment of specificity of immune reaction. Specifically, a detection method by a flow-through method (as described in non-patent document 1 or patent document 1) or an immunochromatography method (a lateral flow system and a tangent flow system, as described in patent documents 2 and 3) has rapidly become prevailed in terms of its simplicity.

A commercially available immunochromatography method is provided with a strip-form membrane, in which a capture reagent (for example, an antibody) to catch a detected substance (for example, an antigen) is fixed on one end of a membrane in its length direction and on the other end, a labeled probe [for example, (1) visible, colloidal gold particles, as described in patent document 4; (2) dyed synthetic polymer latex particles, as described in patent document 5] is held so as to be developable on the membrane. When a specimen containing a detected substance is provided in a prescribed amount on the membrane of the side holding a labeled probe and the specimen is chromatographically developed on the membrane, the substance to be detected combines with the labeled probe to form a complex of the substance to be detected and the labeled probe. The formed complex of the substance to be detected and the labeled probe is developed on the membrane and caught by a capture reagent fixed on the membrane to form a complex of the catching agent, the substance to be detected and the labeled probe at the fixed position of the catching agent. Then, the labeled probe is detected in an appropriate manner (in case of visible gold colloidal particles, for example, their coagulated image), whereby the presence of a substance to be detected can be judged.

However, employing either the visible gold colloid particles or the dyed synthetic polymer latex particles in the immunochromatography resulted in reduced sensitivity and reproducibility, in which quantitative determination is not feasible but only the presence/absence of a substance to be detected is determined, leading to inefficient performance.

Patent document 1: JP 7-034016B

Patent document 2: JP 7-013640B

Patent document 3: JP 2890384B

Patent document 4: JP 64-032169A

Patent document 5: JP 5-010950A

Non-patent document 1: “Guide to Diagnostic Rapid Test Device Components”, 2nd edition, published by Scheicher & Schuell company, January 2000, Edited by Lisa Vickers, pp. 6-8

### DISCLOSURE OF THE INVENTION

### Problem to be Solved

It is an object of the present invention to provide a method for detecting a substance such as a virus, which achieves enhanced detection sensitivity even in trace amounts of a substance to be detected and is also simple and superior in visual determinability, and a detection kit used therein.

### Means for Solving the Problem

As a result of extensive study by the inventors of this application, it was found that the use of semiconductor nanoparticle-labeled probe enables achievement of enhanced detection sensitivity even in trace amounts of a substance to be detected and is superior in simplicity and visual determinability, whereby the present invention has come into being.

Thus, the present invention is specified of constituents, as described below.

One aspect of the invention is directed to a detection method, comprising contacting a complex of a substance to be detected and a semiconductor nanoparticles-labeled probe capable of bonding the substance with an immobilized capture reagent capable of binding to the substance to detect the substance.

Another aspect of the invention is also directed to a detection kit used in the foregoing detection method.

The semiconductor nanoparticles may contain at least one element selected from the group consisting of B, C, N, Al, Si, P, S, Zn, Ga, Ge, As, Se, Cd, In, Sb and Te and preferably exhibit a specific gravity of not more than 3.

The semiconductor nanoparticles preferably exhibit an average particle size of 1 to 50 nm.

The detection method of the invention may suitably employ an immunochromatography method.

A combination of the substance to be detected with the semiconductor nanoparticles-labeled probe may be a combination of an antigen with an antibody or a combination of an antibody with an antigen, and a combination of the substance to be detected with an immobilized capture reagent may be a combination of an antigen with an antibody or a combination of an antibody with an antigen.

The substance to be detected may be a protein derived from a virus.

The virus may be an influenza virus type A, B or C, a norovirus, a SARS (serious acute respiratory syndrome) virus, a hepatitis A virus, hepatitis B virus or hepatitis C virus, a human immunodeficiency virus (HIV), an aftosa virus, or a highly pathogenic avian influenza.

### EFFECT OF THE INVENTION

In the present invention, there can be provided a detection method of a substance such as virus by the use of a probe labeled with semiconductor nanoparticles of a long emission life-time which achieves enhanced detection sensitivity even in trace amounts of a substance to be detected and is superior in simplicity and visual determinability, and a detection kit usable in this method. The invention can provide a more rapid detection method by employment of an immunochromatography method.

### BRIEF DESCRIPTION OF THE DRAWINGS

FIG. 1a shows a plan view of an immunochromatography strip and FIG. 1b shows a vertically sectional view of the immunochromatography strip shown in FIG. 1a.

### DESCRIPTION OF DESIGNATION

1: Adhesive sheet
2: Impregnated member
3: Membrane support
31: Capture site
4: Absorption member
5: Member for sample addition

### PREFERRED EMBODIMENTS OF THE INVENTION

There will be described the present invention in detail.

The present invention is related to a detecting method in which a complex of a substance to be detected and a probe labeled with semiconductor nanoparticles and capable of bonding the substance is brought into contact with an immobilized capture reagent capable of binding to the substance, whereby the substance is detected.

### Substance to be Detected:

In the invention, substances to be detected are not specifically restricted and examples thereof include a protein, a polypeptide, a nucleic acid, a sugar chain, a virus, a cell and the like and of these, one which is capable of becoming an antigen is preferred. Specific examples thereof include proteins derived from a pathogenic virus, such as a protein derived from an influenza virus type A, B or C (for example, hemagglutinin, neuraminidase, M2 protein, ribonucleic acid protein, and the like), and pathogenic viruses such as a norovirus, a SARS (serious acute respiratory syndrome) virus, a A-type, B-type or C-type hepatitis virus, a human immunodeficiency virus (HIV), and an aftosa virus; and pathogenic viruses capable of infecting animals except humans (for example, foot-and-mouth disease virus, highly pathogenic avian influenza, or the like). Among these, it is preferably applicable to a protein derived from an influenza virus type A, B or C.

### Semiconductor Nanoparticle-Labeled Probe:

The semiconductor nanoparticle-labeled probe used in the detection method of the invention refers to a probe which is labeled with semiconductor nanoparticles; the semiconductor nanoparticles are quantum dots exhibiting band-gap emission through high quantum efficiency and are each a particle formed of some hundreds to some thousands of atoms constituting a semiconductor and having a diameter of some nanometers. The shape of semiconductor nanoparticles can be in a spherical form, a bar form, a plate form or a tube form, and the semiconductor nanoparticles used in the invention are preferably in a spherical form or a quasi-spherical form.

Semiconductor nanoparticles achieve an enhanced fluorescence emission intensity through the quantum size effect and the wavelength of emitted fluorescence is variable by a particle's size (herein, the particle size refers to the maximum diameter of semiconductor nanoparticles). Unlike conventional fluorescence dyes, exposure to light having an energy larger than the band gap can achieve efficient excitation irrespective of wavelength of the exposure light. Further, semiconductor nanoparticles exhibit an excellent light absorption characteristic and can be excited by a light source of low luminance, such as a light emission diode (LED). Accordingly, a single exciting light can excite plural semiconductor nanoparticles, whereby multi-color imaging can be simply realized.

When the surfaces of the semiconductor nanoparticles are exposed, a number of defects on the surfaces act as an emission killer, resulting in a reduced emission intensity, so that the semiconductor nanoparticles used in the invention are preferably shelled. Such shelled semiconductor nanoparticles have a core/shell structure, or a so-called double structure, in which the surface of a nanoparticle forming a core portion is covered with the layer of a shell portion. The material forming the shell portion is preferably a compound of groups of II to VI of the periodic table. The foregoing core/shell structure needs to be composed of the composition so that the band gap of the shell portion is larger than that of the core portion. Further, the core portion preferably is a single crystal, whereby, in the case of fine phosphor particles, for example, an optical element of high emission efficiency can be obtained.

Specifically, examples of semiconductor nanoparticles include particles containing at least one element selected from the group consisting of B, C, N, Al, Si, P, S, Zn, Ga, Ge, As, Se, Cd, In, Sb and Te. It is preferable to avoid Cd which is an element of extremely high toxicity so that silicon or its compound or germanium or its compound is preferred, and it is more preferred to contain at least Si or Ge element. In semiconductor nanoparticles composed of Si or Ge, the size thereof is reduced to the region capable of causing a quantum confinement effect, whereby the band gap energy thereof is expanded to the visible region, resulting in an emission phenomenon.

In the invention, such semiconductor nanoparticles are not specifically restricted but preferably are constituted of a core portion which is a silicon nucleus and a shell portion which is a layer mainly composed of silicon oxide. The layer mainly composed of silicon oxide refers to a shell layer having a main component of silicon oxide (SiO₂). The silicon nucleus of the core portion preferably is a single crystal. In semiconductor nanoparticles of such a core/shell structure, the excitation energy of Si of the core portion is 1.1 eV and the excitation energy of silicon oxide (SiO₂) of the shell portion is 8 eV and the band gap is larger than that of CdSe/ZnS nanoparticles [shell portion (ZnS): 3.6 eV, core portion (CdSe): 1.7 eV]. Further, silicon/silica type semiconductor nanoparticles have the least adverse effect on the environment and are superior in safety for a living body when applied to a living body.

The foregoing semiconductor nanoparticles may be produced in accordance with techniques known in the art or the methods described in the literature. For instance, JP 5-224261A discloses a preparation method of nanoparticles doped with a solid solution of a rare earth element by a treatment technique in combination with either one of a solution synthesis method and a method of a reaction environment at a temperature which is markedly lower than the fusion temperature of a material. Specifically, there are disclosed a method in which a nano-particulate metal halide compound doped with at least one rare earth element and a method in which nanoparticles are precipitated from an aqueous solution of a rare earth element salt and a water-soluble salt of a halide-forming metal. A solution method in which nanoparticles of a rare earth element-doped host substance are prepared from a solution, is specifically desirable.

The production method of semiconductor nanoparticles having a core/shell structure is based on techniques known in the art or methods described in the literature. For instance, synthesis of nano-composite particles having a structure of a silicon dioxide (SiO₂) shell and a metal core was first reported by Mulvaney et al. (Langmuir, 12: 4329-4335, 1996) or Adair et al. (Materials Sci. & Eng. R. 23: 139-242, 1998). SiO₂-coated semiconductor nanoparticles having a core/shell structure are mostly classified into two categories.

The method disclosed in Mulvaney et al. required surface modification of a metal cluster core with a silane coupling agent, 3-aminopropylethoxysilane (APS) before forming a silica shell. The APS was used as an accelerating agent for adhesion between SiO₂ and the metal cluster core which was deficient in affinity to a glassy material. Adair et al. succeeded in coating a metal and CdS cluster with SiO₂ through a simple hydrolysis and condensation of tetraethoxysilane (TEOS) in a cyclohexane/Igepal/water three-component system having an aqueous phase. In this system, small water droplets are enclosed in oil, whereby the thicknesses of both the core and the shell can be controlled, enabling extremely uniform silica-shell coating.

The foregoing semiconductor nanoparticles preferably exhibit a specific gravity of not more than 3, and more preferably not more than 2.5. Semiconductor nanoparticles having a specific gravity of not more than 3 do not easily precipitate and are diffused in the solution, promoting reactivity (contact probability). A specific gravity of not more than 2.5 further enhances diffusibility.

The average particle size of the semiconductor nanoparticles preferably is from 1 to 50 nm, and more preferably from 1 to 20 nm. The semiconductor nanoparticles with an average particle size of 1-50 nm excel in diffusibility in solution and rarely cause steric hindrance. Further, an average particle size of 1 to 20 nm enables effective bonding of a label to a biomolecule, leading to enhanced detection precision.

Examples of the probe of a semiconductor nanoparticle-labeled probe include a monoclonal antibody, polyclonal antibody, fatty acid, enzyme/antibody, avidin/biotin, ribonucleic acid, deoxyribonucleic acid and their oligomers. Of these, an antigen is preferred. An antigen is strong in bonding in the equilibrium state and exhibits enhanced specificity and general-usage.

In cases when a substance to be detected is an antibody, the probe may be an antigen which the antibody recognizes. Thus, the combination of the substance to be detected and the semiconductor nanoparticle-labeled probe preferably is a combination of an antibody and an antibody, or a combination of an antibody and an antigen.

There are known many methods of allowing semiconductor nanoparticles to label a probe. A specific example of a method of allowing SiO₂-shelled semiconductor nanoparticles to label a probe is shown as follows. SiO₂-covered semiconductor nanoparticles are reacted with 3-aminopropylethoxysilane (product of Pierce Co.) and subsequently, SMCC [succinimidyl-4-(N-maleimidomethyl)cyclohexane-1-carboxylate] activation is performed. A thiol group attached to a protein required to react with the thus activated semiconductor nanoparticles can be formed by allowing a protein containing a lysine residue to react with 2-iminothiorane. In this reaction, a lysine side-chain of the protein to be attached reacts with 2-iminothiorane along with ring opening and formation of thioamidine. Subsequently the formed thiol group covalent-bonded to the protein reacts with a maleimido group bonded to the semiconductor nanoparticle surface, thereby forming a covalent bond between an antibody as a protein and a reactive group existing on the surfaces of the foregoing SiO₂-covered nanoparticles.

The semiconductor nanoparticle-labeled probe used in the invention preferably is a monoclonal antibody labeled with shelled silicon (or shelled Si), a monoclonal antibody labeled with shelled germanium (shelled Ge), or a polyclonal antibody labeled with shelled Ge.

### Immobilized Capture Reagent:

An immobilized capture reagent used in the invention is not limited to the following but, for example, in an immunochromatography method described later, refers to a reagent fixed on the strip-form membrane used for capture of a complex of a substance to be detected and the foregoing labeled probe. The capture reagent can be bound to the substance to be detected.

Specific Examples of the immobilized capture reagent include a monoclonal antibody, a polyclonal antibody, a fatty acid, enzyme/antigen, avidin/biotin, deoxyribonucleic acid, deoxyribonucleic acid or their oligomers. Of these, an antibody is preferred. An antibody is preferred, which exhibits enhanced bond strength and is high in specificity and broad utility.

In the case of the substance to be detected being an antibody, the capture reagent may be an antigen recognized by the antibody. Thus, the combination of the substance to be detected and the immobilized capture reagent preferably is a combination of an antigen and an antibody, or a combination of an antibody and an antigen.

### Detection Method

In the detection method of the invention, a test substance containing the substance to be detected is collected from saliva, sweat, urine, blood (whole blood, blood serum/blood plasma) or other humors, which may optionally be pre-treated with a sample dissolving solution, a diluting solution, a buffer solution, a washing solution or the like. Detection is conducted in the following manner. First, a test substance, as described above is mixed with a semiconductor nanoparticle-labeled probe to be bonded to each other. Then, the thus bonded complex is separated from the free-labeled probe, if necessary, by a treatment such as washing. The complex is exposed to an exciting light to detect fluorescence from the semiconductor nanoparticles. Detection may be performed by visual observation or by using an instrument to quantitative-determine the fluorescence amount.

An embodiment of employing an immunochromatography method is preferred to perform simple and rapid detection, as described above.

### Immunochromatography Method:

An immunochromatography method (hereinafter, also denoted as an immunochromato method) can be readily conducted in accordance with configuration of an existing test strip for an immunochromatography method (hereinafter, also denoted simply as an immunochromato strip).

In general, an immunochromato strip is provided with a first antibody capable of undergoing an antibody-antigen reaction at a first antigen-determining group of an antigen, a second antibody capable of performing an antibody-antigen reaction and labeled at a second antigen-determining group of the antigen and a membrane support, in which the first antibody is preliminarily fixed at the prescribed position of the membrane support to form a capture-site and the second antibody is disposed (for example, in an immersion form) at a position spaced from the capture site so that chromatography is developable on the membrane support.

The foregoing first antibody and second antibody, each may be a polyclonal antibody or a monoclonal antibody, but at least one of them preferably is a monoclonal antibody. Usually, the first antibody and the second antibody are used in a “heterogeneous” combination, that is, the first antibody and the second antibody, each of which recognizes the respective antigen-determining groups differing in position and structure on the antigen, are used in combination. However, the first antigen-determining group and the second antigen-determining group may be the same in structure if they differ in the position on the antigen. In that case, the first antibody and the second antibody may be monoclonal antibodies in a homogeneous combination, that is, an identical monoclonal antibody is usable in both of the first antibody and the second antibody.

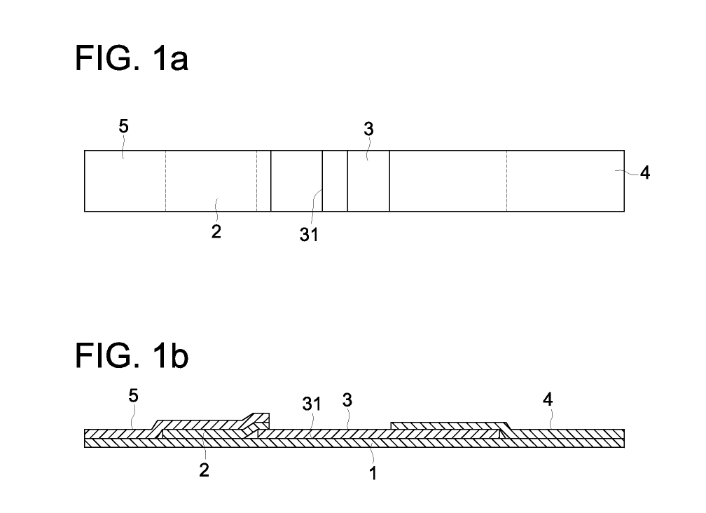

A specific example of an immunochromato strip is shown in FIGS. 1a and 1b, in which the numeral 1 represents a adhesive sheet, the numeral 2 represents an impregnated member, the numeral 3 represents a membrane support, the numeral 31 represents a capture site, the numeral 4 represents an absorption member, and the numeral 5 represents a member for sample addition.

Examples of FIGS. 1a and 1b include the embodiment described below.

As a membrane support (3) of an immunochromato strip is prepared a long belt-form nitrocellulose membrane filter with a 5 mm width and a 36 mm length.

A first antibody (for example, immobilized capture reagent) is fixed at a position of 7.5 mm from the end on the side of initiation point of chromatography development on the membrane support (3), whereby a capture site (31) is prepared.

The membrane support (3) employs a nitrocellulose membrane filter but there may be used any one in which a substance to be detected is chromatographically developable and the first antibody forming the capture site (31) is fixable, and other cellulose membranes, a nylon membrane, a glass fiber membrane or the like is usable.

The impregnated member (2) is comprised of a member impregnated with the second antibody (for example, a semiconductor nanoparticle-labeled probe) which recognizes the second antigen-determining group existing at a site differing from that of the first antigen-determining group. The second antibody is preferably labeled in advance with an appropriate labeling substance.

Examples of a material used for the impregnated member (2) include a glass-fiber nonwoven fabric, a cellulose fabric (filter paper, nitrocellulose membrane, or the like), and a porous plastic fabric such as polyethylene or polypropylene.

To add a test substance to a substance to be detected, the member for sample addition (5) may be provided at the end of the foregoing immunochromato strip in the direction opposite the side to develop chromatography of the impregnated member (2). Further, to absorb a test substance which has been chromatographically developed, the absorption member (4) is desirably provided at the end of the immunochromato strip in the direction of chromatographically developing the impregnated member (2). Chromatographical development is promoted by providing the absorption member (4).

The immunochromato strip is not limited to one, as described above but may be appropriately deformed or changed.

### Detection Kit:

A detection kit used in the detection method of the invention include, as an essential constituent element, a support containing semiconductor nanoparticle-labeled probe capable of bonding to a substance to be detected, such as a microplate (for example, 96-hole microplate), affinity beads or an immunochromato strip, and optionally includes a dissolution solution to dissolve a test substance, a reaction reagent and a detection reagent. Further, there may be included various equipments, materials or reagents necessary for practice of the method of the invention. Such reagents may include a sample-dissolving solution, a diluting solution, a buffer solution, a washing solution, a reaction-stopping agent, a (product) extracting solution and the like.

Further, constituent elements of the detection kit may include a reference material to prepare a calibration curve, an explanatory leaflet and a set of equipments and materials, such as a microplate capable of simultaneous-processing plural test substances and a plate reader as a detection device thereof. A preferred aspect of a detection kit used in the examination method of the invention include an immunochromato strip capable of bonding to a substance to be detected and using a semiconductor nanoparticle-labeled antibody, a diluting solution for a test substance and a light source to excite semiconductor nanoparticles.

### EXAMPLES

The present invention will be described in detail with reference to examples, but the invention is by no means limited to these.

### Example 1

### Preparation of Si Nanoparticles

To 50 ml of dioctyl ether were added 1 ml of oleic acid and 1 ml of oleyl amine, stirred, and then heated to 100° C. with degassing. After stirring for 3 hours, the reaction mixture was heated to 200° C., while filling the reaction vessel with argon. After stirring for another 1 hour, 1 ml of SiCl₄ was added dropwise over 30 seconds and was stirred for 30 minutes. The reaction mixture was cooled to 100° C., stirred for 5 hours and then cooled to room temperature to obtain Si nanoparticles. The specific gravity of the obtained Si nanoparticles was 2.3 and the average particle size was 3.0 nm.

### Preparation of Si Nanoparticle-Labeled Antibody:

First, lithium aluminum hydride as a reducing agent and allyl amine were added to the obtained Si nanoparticles and mixed in dioctyl ether to obtain Si nanoparticles having amino groups as a surface functional group. This solution was filtered to obtain particles, which were washed and dried. The thus obtained Si nanoparticles were dispersed in an aqueous solution exhibiting a pH of 5.0. The dispersion was subjected to conversion reaction to allow a thiol-reactive maleimide group to be attached to the foregoing amino groups. Namely, using 4-maleimidomethyl)-1-cyclohexanecarboxylic acid N-hydroxysuccinimide ester (SMCC) as a divalent cross-linker reagent, the reaction was performed and then, gel filtration chromatography was conducted over 60 minutes, whereby excessive cross-linkers were removed from the Si nanoparticles.

Subsequently, in order to allow an antibody (complete IgG molecule) to yield a thiol group to label Si nanoparticles activated by an maleimido group, as described above, the antibody was treated with dithiothreitol (DTT) to reduce a disulfide bonding which was inherently held by the antibody. After this reaction, gel filtration chromatography was performed to remove excessive reducing reagent, DTT. The thus obtained antibody having a thiol group and the foregoing maleimido group-activated Si nanoparticles were reacted to allow a silicon nanoparticle to be attached onto the antibody surface. Further, 2-mercaptoethanol was added thereto to block extraneous maleimido groups which did not participate in the reaction.

Finally, size exclusion chromatography (SEC) was conducted by employing a column filled with Superdex(R) 200 to separate antibodies not labeled with Si nanoparticles from the Si nanoparticle-labeled antibodies. Namely, a column was filled with Superdex(R) 200 and then equilibrated with a phosphate-buffered sodium chloride solution (PBS), and the foregoing mixture which was concentrated by ultrafiltration was loaded to the column. The mixture was eluted by the PBS and finally, a 100-120 μL solution were recovered. The finally obtained solution, which contained no unlabeled antibody, was diluted to an optimal concentration for use in the examples described below.

### Preparation of Immunochromato Strip:

A long belt-form nitrocellulose filter of a 5 mm width and a 40 mm length was prepared as a membrane support for use in the chromatography development. An amount of 0.1% of a buffer solution of anti-influenza virus type A monoclonal antibody was replaced by a 10 mM trehalose citric acid buffer solution and an optimum quantity thereof was added dropwise into the end of the membrane support and dried to form a capture site.

A 5×15 mm belt-form glass fiber unwoven fabric was impregnated with a Si nanoparticle-labeled antibody and dried to prepare a member impregnated with a Si nanoparticle-labeled antibody.

Next, a cotton fabric as a member for sample addition, the member impregnated with a Si nanoparticle-labeled antibody, the membrane support for use in chromatography development and a belt-form filter paper as a absorption member were each adhered to a prescribed position on the adhesive surface of a belt-form adhesive sheet to prepare an immunochromato strip.

### Measurement by Immunochromato Method:

A commercially available nucleoprotein derived from influenza type A was diluted with a developing solvent (PBS) to 1800 times, 1000 times and 500 times, respectively to prepare diluted test substance solutions. Further, a developing solvent was prepared as a blank solution. Only each of the diluted test substance solutions and a developing solvent were dropwise added onto the member for sample addition. Liquid was developed and there was visually observed a red fluorescence emitted from the membrane member at the portion to which the anti-influenza virus type A monoclonal antibody was adsorbed, when exposed to a 350 nm exiting light source. The presence of an influenza virus type A in the diluted test substance solution was confirmed through red fluorescence emission. When no change in color occurred and the color of the membrane support was observed, it indicated that no influenza type A virus was present in a sample.

The results are shown in Table 1. The designations shown in the Table are as follows:

+++: Coloring was observed at a strong level,

++: Coloring was observed at a medium level,

+: Coloring was slightly observed,

−: No coloring was observed.

### Example 2

### Preparation of CdSe Nanoparticles

Into a round-bottom flask were placed 1 g of selenium pellets and 11.3 g of trioctylphosphine and stirred at 150° C. for 1 hour under an Ar atmosphere. Thereto, 38.6 g of trioctylphosphine oxide was added and heated at 80° C. for 40 minutes to remove the Ar. Thereafter, 3.9 g of cadmium acetate dihydride was added and stirred at 80° C. for 4 hours with removing Ar gas to obtain CdSe nanoparticles. The specific gravity of the thus obtained CdSe nanoparticles was 6.8 and the average particle size was 4.0 nm.

Similarly to Example 1, measurement was conducted by the immunochromato method, provided that CdSe nanoparticles were used in place of Si nanoparticles. A 350 nm exciting light was used for visual examination.

The results thereof are shown in Table 1.

### Comparative Example 1

### Preparation of Colloidal Gold Particles

Ultrapure 99 ml water was boiled, 1 ml of an aqueous chloroauric acid solution was added thereto, and 1.5 ml of an aqueous 1% by mass sodium citrate solution was further added and refluxed. Thereafter, the mixture was allowed to stand at mom temperature to prepare a suspension.

### Preparation of Colloidal Gold-Labeled Antibody

To the obtained suspension was added an aqueous potassium carbonate solution to adjust the pH to 7.6. Anti-influenza virus type A monoclonal antibody which was preliminarily purified by dialysis and centrifugal separation was added to a boric acid solution in an amount of 10 μg per ml of boric acid solution and thereto the foregoing colloidal gold suspension was added with stirring. Further thereto was added 0.1 ml of BSA at a concentration of 30% by mass and stirred by a rotator. The total amount thereof was subjected to centrifugal sedimentation, and to precipitated colloidal gold and one sensitized with anti-influenza virus type A monoclonal antibody was added 1 ml of a mixture of a 50 mM tris-hydrochloric acid buffer solution, 1% BSA and 200 mM sodium chloride.

Similarly to Example 1, measurement was conducted by the immunochromato method, provided that colloidal gold were used in place of Si nanoparticles. When a membrane member, a portion onto which was adsorbed anti-influenza virus type A monoclonal antibody was colored with colloidal gold, it confirmed the presence of influenza virus type A virus in a sample. When no change in color occurred, while the color of the membrane support was observed, it indicated that no influenza type A virus was present in a sample.

The results thereof are shown Table 1.

### Comparative Example 2

Similarly to Comparative Example 1, measurement was conducted by the immunochromato method, provided that FITC (fluorescein isothiocyanate) were used in place of colloidal gold, and fluorescence was read by a fluorescence reader (Jenios Pro, produced by Cosmo Bio Co.).

The results thereof are shown Table 1.

TABLE 1

| | | | | Comparative | Comparative |
|---|---|---|---|---|---|
| | | Example 1 | Example 2 | Example 1 | Example 2 |
| Judgment Method | | Visual | Visual | Visual | Reader |
| Influenza Type A | 1800 times dilution | ++ | + | − | + |
| Nucleoprotein | 1000 times dilution | +++ | ++ | + | + |
| | 500 times dilution | +++ | +++ | + | ++ |

| | | | | |
|---|---|---|---|---|
| Developing Solvent alone | − | − | − | − |

In the method and the device according to the present invention, judgment could be made visually in a simple manner without requiring any dedicated detector, as in case of a fluorescent dye, and enhanced detection sensitivity was achieved even in a trace amount of a substance to be detected, as compared to the use of gold colloid. As can be seen from comparison of Examples 1 and 2, superior results were achieved when using Si particles having a specific gravity of not more than 3. It is supposed that the difference in specific gravity resulted in different specificities.

1. A detection method comprising contacting a complex of a substance to be detected and a semiconductor nanoparticles-labeled probe capable of bonding the substance with an immobilized capture reagent capable of bonding the substance to detect the substance.

2. The detection method as claimed in claim 1, wherein the semiconductor nanoparticles contain at least one element selected from the group consisting of B, C, N, Al, Si, P, S, Zn, Ga, Ge, As, Se, Cd, In, Sb and Te.

3. The detection method as claimed in claim 2, wherein the semiconductor nanoparticles exhibit a specific gravity of not more than 3.

4. The detection method as claimed in claim 1, wherein the semiconductor nanoparticles preferably exhibit an average particle size of 1 to 50 nm.

5. The detection method as claimed in claim 1, wherein the detection method is an immunochromatography method.

6. The detection method as claimed in claim 1, wherein a combination of the substance to be detected and the labeled probe is a combination of an antigen and an antibody or a combination of an antibody and an antigen.

7. The detection method as claimed in claim 1, wherein a combination of the substance to be detected and the immobilized capture reagent is a combination of an antigen and an antibody or a combination of an antibody and an antigen.

8. The detection method as claimed in claim 1, wherein the substance to be detected is a protein derived from a virus.

9. The detection method as claimed in claim 8, wherein the virus is an influenza virus type A, B or C, a norovirus, a SARS (serious acute respiratory syndrome) virus, a hepatitis A virus, hepatitis B virus or hepatitis C virus, a human immunodeficiency virus (HIV), an aftosa virus, or a highly pathogenic avian influenza.

10. A detection kit used in a detection method comprising contacting a complex of a substance to be detected and a semiconductor nanoparticles-labeled probe capable of bonding the substance with an immobilized capture reagent capable of bonding the substance to detect the substance.
